(19) United States
(12) Patent Application Publication
(10) Pub. No.: US 2025/0369025 A1
(43) Pub. Date: Dec. 4, 2025

(54) PRODUCTION METHOD OF A PHOSPHOLIPID

(71) Applicant: NIPPON FINE CHEMICAL CO., LTD., Osaka (JP)

(72) Inventors: Yuki Teshima, Takasago-shi (JP); Hironobu Ishiyama, Takasago-shi (JP); Takara Hagimori, Takasago-shi (JP); Tomoyuki Koge, Takasago-shi (JP)

(73) Assignee: NIPPON FINE CHEMICAL CO., LTD., Osaka (JP)

(21) Appl. No.: 19/082,966

(22) Filed: Mar. 18, 2025

(30) Foreign Application Priority Data
May 28, 2024 (JP) 2024-086286
Nov. 28, 2024 (JP) 2024-207482

(51) Int. Cl. C12P 7/6481

(52) U.S. Cl. CPC C12P 7/6481

(57) ABSTRACT

A method for producing a phospholipid represented by formula (1) according to reaction equation I,

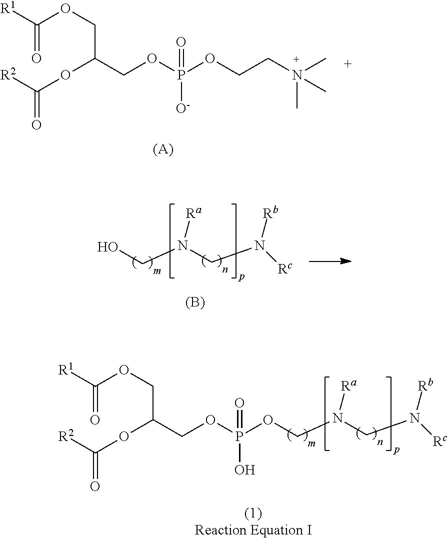

wherein R¹ and R² are the same or different and each represents an acyclic hydrocarbon group; R^{a}, R^{b}, and R^{c} are the same or different and each represents a hydrogen atom or a C₁-C₅ hydrocarbon group; R^{a} and R^{b} may be linked together to form a ring containing adjacent nitrogen atoms; m represents 1 or 2; n represents 1 or 2; and p represents an integer of 1 to 4,
the method comprising:
mixing an organic solvent solution containing a compound represented by formula (A) with an aqueous solvent solution containing phospholipase D and a compound represented by formula (B) in a flow channel; and
reacting the mixture at an enzyme reaction temperature of phospholipase D.

### TECHNICAL FIELD

The present invention relates to a method for producing a phospholipid, for example.

### BACKGROUND ART

Recent years have seen enormous promise in RNA interference agents, including small interfering RNA (siRNA), as fascinating pharmaceutical seeds. Although a stream of high-potential seeds have been discovered, an extremely sophisticated delivery system is required for externally administered RNA to exhibit its intrinsic activity in vivo. This is because RNA is readily enzymatically degraded by nucleases, and poorly penetrates cell membranes. Thus, the commercial viability of RNA interference agents inevitably involves the development of a delivery system.

A known delivery system for medicinal substances, such as RNA, is the administration of a medicinal substance encapsulated in a lipid particle. However, administering a negatively charged nucleic acid typically involves the use of a positively charged lipid to cause electrostatic interaction; this raises concerns regarding cytotoxicity (PTL 1).

Under such circumstances, PTL 2 reports on a phospholipid (a phospholipid represented by formula (1) described later) that is not positively charged at the pH of bodily fluids (usually in the neutral range) and that can form lipid particles capable of more efficiently exerting the effect of the encapsulated drug.

### CITATION LIST

### Patent Literature

PTL 1: JP2016-023147A
PTL 2: WO2018/190017

### SUMMARY OF INVENTION

### Technical Problem

The present invention aims to provide a method for more efficiently producing a phospholipid represented by formula (1).

### Solution to Problem

As a result of extensive study in view of the above object, the present inventors found that when two types of solutions (an organic solvent solution and an aqueous solvent solution) each containing a raw material of a phospholipid represented by formula (1) are mixed together in a flow channel and then reacted at an enzyme reaction temperature of phospholipase D, the reaction time can be shortened and the phospholipid represented by formula (1) can be produced more efficiently. As a result of further study based on this finding, the present inventors completed the present invention. Specifically, the present invention encompasses the following embodiments.

Item 1. A method for producing a phospholipid represented by formula (1) according to reaction equation I

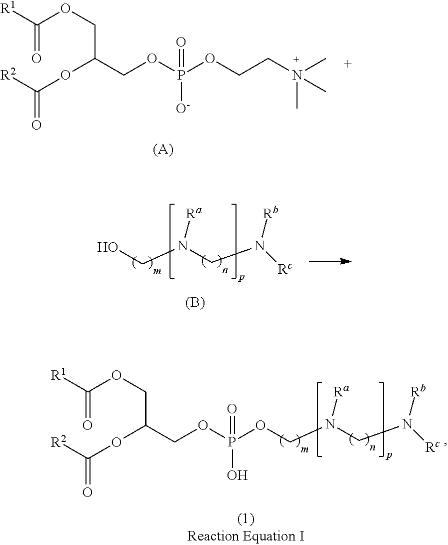

wherein R¹ and R² are the same or different and each represents a acyclic hydrocarbon group; R^{a}, R^{b}, and R^{c} are the same or different and each represents a hydrogen atom or a C₁-C₅ hydrocarbon group; R^{a} and R^{b} may be linked together to form a ring containing adjacent nitrogen atoms; m represents 1 or 2; n represents 1 or 2; and p represents an integer of 1 to 4,
the method comprising:
mixing an organic solvent solution containing a compound represented by formula (A) with an aqueous solvent solution containing phospholipase D and a compound represented by formula (B) in a flow channel; and
reacting the mixture at an enzyme reaction temperature of phospholipase D.

Item 2. The method according to Item 1, wherein a cross-sectional area of the flow channel is 0.01 mm² to 10 mm².

Item 3. The method according to Item 1 or 2, wherein a flow rate per mm² of cross-sectional area of the flow channel is 50 cm³/h to 1000000 cm³/h.

Item 4. The method according to any one of Items 1 to 3, wherein the mixing in the flow channel is carried out using a static mixer.

Item 5. The method according to any one of Items 1 to 4, wherein the organic solvent solution contains an ether solvent and/or an ester solvent.

Item 6. The method according to any one of Items 1 to 5, wherein the aqueous solvent solution contains a buffer.

Item 7. The method according to any one of Items 1 to 6, wherein the reaction is carried out for 240 min or less.

Item 8. The method according to any one of Items 1 to 7, wherein at least a part of the reaction is carried out in a plug flow reactor.

Item 9. The method according to any one of Items 1 to 8, wherein the acyclic hydrocarbon group is an unsaturated acyclic hydrocarbon group.

Item 10. The method according to any one of Items 1 to 9, wherein the acyclic hydrocarbon group has 12 to 24 carbon atoms.

Item 11. The method according to any one of Items 1 to 10, wherein m and n are both 2.

Item 12. The method according to any one of Items 1 to 11, wherein p is 1 or 2.

Item 13. The method according to any one of Items 1 to 12, wherein R^{a}, R^{b}, and R^{c} are hydrogens.

Item 14. The method according to any one of Items 1 to 13, wherein R^{a}, R^{b}, and R^{c} are the same or different and each represents a C₁-C₅ hydrocarbon group, R^{a} and R^{b} may be linked together to form a ring containing adjacent nitrogen atoms.

### Advantageous Effects of Invention

The present invention can provide a method for more efficiently producing a phospholipid represented by formula (1).

### BRIEF DESCRIPTION OF DRAWINGS

FIG. 1 shows results of thin-layer chromatography analysis of reaction solutions of Example 1 and Comparative Example 1. A spot representing DOPC is indicated by an arrow.

### DESCRIPTION OF EMBODIMENTS

Herein, the terms “include,” “contain” and variations thereof express concepts that encompass all of “comprise,” “consist of,” and “consist essentially of.”

In one embodiment, the present invention relates to a method for producing a phospholipid represented by formula (1) according to reaction equation I below

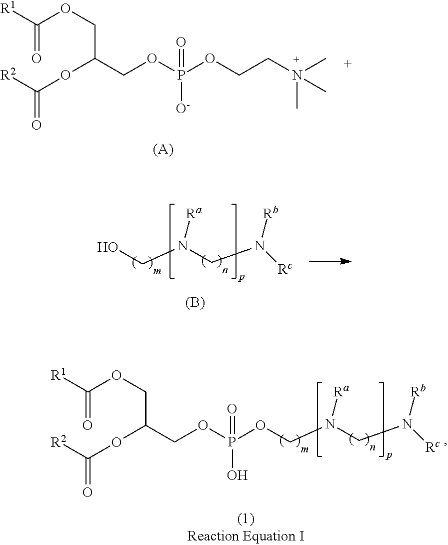

the method comprising: mixing an organic solvent solution containing a compound represented by formula (A) with an aqueous solvent solution containing phospholipase D and a compound represented by formula (B) in a flow channel; and reacting the mixture at an enzyme reaction temperature of phospholipase D (herein, the method is sometimes referred to as “the production method of the present invention”). Hereinafter, the method is described.

R¹ and R² are the same or different and each represents an acyclic hydrocarbon group.

The acyclic hydrocarbon group represented by R¹ or R² is not limited as long as it is a monovalent acyclic hydrocarbon group and includes both linear and branched (preferably linear) hydrocarbon groups. The carbon number of the acyclic hydrocarbon group is not limited, as long as it is sufficient to form lipid particles. For example, the carbon number is 4 to 30, preferably 8 to 26, more preferably 12 to 22, still more preferably 14 to 20, yet still more preferably 15 to 19. The acyclic hydrocarbon group includes both saturated acyclic hydrocarbon groups and unsaturated hydrocarbon groups. The acyclic hydrocarbon group is preferably an unsaturated acyclic hydrocarbon group, more preferably an unsaturated acyclic hydrocarbon group containing a double bond, still more preferably an unsaturated acyclic hydrocarbon group containing only one double bond. Examples of acyclic hydrocarbon groups include butyl, pentyl, hexyl, heptyl, octyl, nonyl, decyl, undecyl, tridecyl, tetradecyl, pentadecyl, 9-pentadecenyl, hexadecyl, heptadecyl, cis-9-heptadecenyl, 11-heptadecenyl, cis, cis-9,12-heptadecadienyl, 9,12,15-heptadecantrienyl, 6,9,12-heptadecantrienyl, 9,11,13-heptadecantrienyl, nonadecyl, 8,11-nonadecadienyl, 5,8,11-nonadecatrienyl, 5,8,11,14-nonadecatetraenyl, henicosyl, tricosyl, cis-15-tricosenyl, pentacosyl, heptacosyl, and nonacosyl.

Preferably at least one of R¹ or R² is an unsaturated acyclic hydrocarbon group. More preferably, both are unsaturated acyclic hydrocarbon groups.

R^{a}, R^{b}, and R^{c} may be the same or different and each may represent a hydrogen atom or a C₁-C₅ hydrocarbon group, and R^{a} and R^{b} may be linked together to form a ring containing adjacent nitrogen atoms.

The C₁-C₅ hydrocarbon group is not limited as long as it is a monovalent hydrocarbon group. The hydrocarbon group is preferably an acyclic hydrocarbon group (more preferably linear), more preferably an alkyl group. The carbon number of the hydrocarbon group is preferably 1 to 3, more preferably 1 or 2, still more preferably 1. Specific examples of hydrocarbon groups include methyl, ethyl, propyl, butyl, and pentyl. Of these, methyl is particularly preferred.

That “R^{a} and R^{b} may be linked together to form a ring containing adjacent nitrogen atoms” means that R^{a} and R^{b} are linked together to form a divalent acyclic hydrocarbon group, with one end of the divalent acyclic hydrocarbon group linked to a nitrogen atom adjacent to R^{a} in formula (1) or (B) and the other end linked to a nitrogen atom adjacent to R^{b} in formula (1) or (B).

The divalent acyclic hydrocarbon group is preferably linear, more preferably an alkyl group. The carbon number of the acyclic hydrocarbon group is preferably 1 to 8, more preferably 1 to 5, still more preferably 1 to 3, yet still more preferably 2 or 3, particularly preferably 2.

When R^{a} and R^{b} are linked together to form a ring and p is 2 to 4 (i.e., there are multiple R^{a}s), the Ra forming the ring is any one of these R^{a}s, preferably the Ra closest to —NR^{b}R^{c}.

In one embodiment of the present invention, preferably, Ra, Rb, and Rc are all hydrogen atoms.

In one embodiment of the present invention, preferably, R^{a}, R^{b}, and R^{c} are all alkyl groups, particularly preferably methyl groups.

In one embodiment of the present invention, preferably, R^{a}, R^{b}, and R^{c} are the same or different and each represents a C₁-C₅ hydrocarbon group, and R^{a} and R^{b} may be linked together to form a ring containing adjacent nitrogen atoms.

m represents 1 or 2. Preferably, m is 2.

n represents 1 or 2. Preferably, n is 2.

Preferably, m and n are both 2.

p represents an integer of 1 to 4. Preferably, p is 1 or 2. p is more preferably 1 in terms of phospholipid cytotoxicity. p is more preferably 2 in terms of encapsulation efficiency of a drug in lipid particles derived from phospholipids.

The following moiety in formula (1) and (B)

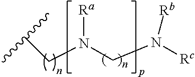

includes the following four preferred embodiments:

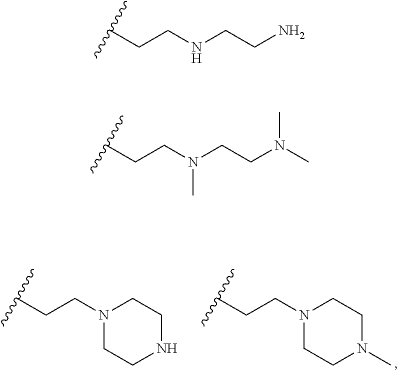

with the following two embodiments being particularly preferred:

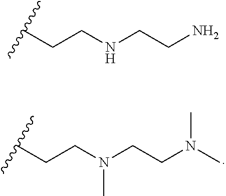

Any organic solvent solution can be used as long as it is a solution containing a compound represented by formula (A) and an organic solvent as a main solvent (e.g., the organic solvent content is 70 vol % or more, preferably 80 vol % or more, more preferably 90 vol % or more, still more preferably 95 vol % or more, yet still more preferably 99 vol % or more, particularly preferably 100 vol %, relative to a total 100 vol % of the solvent constituting the organic solvent solution).

Any organic solvent can be used as long as it is a solvent that can dissolve the compound represented by formula (A) and that has a boiling point in at least a temperature range in which phospholipase D can exert its enzymatic activity. Examples of organic solvents include ether solvents, ester solvents, halogen solvents, hydrocarbon solvents, and aromatic hydrocarbon solvents. Of these, ether solvents and ester solvents are preferred, ether solvents are more preferred, and hydrophobic ether solvents are still more preferred. These may be used alone or in a combination of two or more.

Examples of ether solvents include cyclopentyl methyl ether, 4-methyltetrahydropyran, tetrahydrofuran, 2-methyltetrahydrofuran, 1,4-dioxane, diethyl ether, diisopropyl ether, t-butyl methyl ether, and tetrahydropyran. Of these cyclopentyl methyl ether and 4-methyltetrahydropyran are particularly preferred.

Examples of ester solvents include ethyl acetate, methyl acetate, butyl acetate, propyl acetate, and methyl lactate.

Examples of halogen solvents include chloroform, dichloromethane, and carbon tetrachloride.

Examples of hydrocarbon solvents include hexane, pentane, octane, nonane, and decane.

Examples of aromatic hydrocarbon solvents include benzene, toluene, and xylene.

The concentration of the compound represented by formula (A) in the organic solvent solution is not limited. For example, it is 0.01 g/mL to 1 g/mL, preferably 0.03 g/mL to 0.5 g/mL, more preferably 0.08 g/mL to 0.2 g/mL.

The organic solvent solution can be obtained by mixing the compound represented by formula (A) with an organic solvent.

Any aqueous solvent solution can be used as long as it is a solution containing phospholipase D, a compound represented by formula (B), and water as a main solvent (e.g., the water content is 70 vol % or more, preferably 80 vol % or more, more preferably 90 vol % or more, still more preferably 95 vol % or more, yet still more preferably 99 vol % or more, particularly preferably 100 vol %, relative to a total 100 vol % of the solvent constituting the aqueous solvent solution).

Phospholipase D is an enzyme that can hydrolyze phosphatidylcholine into phosphatidic acid and choline. For example, it is an enzyme classified as phosphatidylcholine phosphatidohydrolase (EC 3.1.4.4).

Phospholipase D may be derived from any organism, preferably a bacterium, particularly preferably a bacterium of the genus Streptomyces.

In terms of yield and the like, phospholipase D is used in an amount of preferably 100 to 1500 U, more preferably 250 to 1000 U, still more preferably 300 to 600 U, per mml of the compound represented by formula (A). One unit (1 U) is defined as the amount of enzyme that can convert 1 micromole (μmol) of substrate per minute (1 μmol per minute) under optimal conditions (temperature of 30° C. and acidity at which the chemical reaction proceeds to the greatest extent).

In terms of yield and the like, the compound represented by formula (B) is used in an amount of preferably 2 to 20 mol, more preferably 4 to 16 mol, still more preferably 5 to 10 mol, per mole of the compound represented by formula (A).

The concentration of phospholipase D in the aqueous solvent solution is not limited. For example, it is 0.05 mg/mL to 5 mg/mL, preferably 0.15 mg/mL to 2 mg/mL, more preferably 0.20 mg/mL to 1 mg/mL.

The concentration of the compound represented by formula (B) in the aqueous solvent solution is not limited. For example, it is 0.02 g/mL to 2 g/mL, preferably 0.05 g/mL to 1 g/mL, more preferably 0.08 g/mL to 0.5 g/mL.

Preferably, the aqueous solvent solution contains a buffer. Examples of buffers include acetate buffers, phosphate buffers, citrate buffers, succinate buffers, and phthalate buffers. Of these, acetate buffers are particularly preferred.

The pH of the aqueous solvent solution is preferably 4 to 7, more preferably 5 to 6.

In addition to the above-described components, other appropriate additives can be added to the organic solvent solution and the aqueous solvent solution for the reaction within the range that does not significantly impair the progress of the reaction. The amount of such additives in each solution is, for example, 10 parts by mass or less, 5 parts by mass or less, 2 parts by mass or less, 1 part by mass or less, 0.1 parts by mass or less, 0.01 parts by mass or less, 0.001 parts by mass or less, 0.0001 parts by mass or less, or 0.00001 parts by mass or less, relative to, for example, 100 parts by mass of the compound represented by formula (A).

In the production method of the present invention, the organic solvent solution is mixed with the aqueous solvent solution in the flow channel. This allows the organic solvent solution and the aqueous solvent solution to be suitably mixed while the activity of phospholipase D is maintained at a certain level or higher by turbulence, backflow, crossflow, and the like that occur in the flow channel, thus achieving high reaction efficiency.

In terms of reaction efficiency, the cross-sectional area of the flow channel is preferably 0.01 mm² to 10 mm², more preferably 0.1 mm² to 10 mm², still more preferably 0.3 mm² to 5 mm², yet still more preferably 0.7 mm² to 1.8 mm².

In terms of reaction efficiency, the flow rate per mm² of cross-sectional area of the flow channel is preferably 50 cm³/h to 1000000 cm³/h, more preferably 50 cm³/h to 100000 cm³/h, still more preferably 50 cm³/h to 10000 cm³/h, yet still more preferably 50 cm³/h to 5000 cm³/h, further more preferably 50 cm³/h to 1500 cm³/h, still further more preferably 150 cm³/h to 1000 cm³/h, particularly preferably 300 cm³/h to 600 cm³/h.

In terms of reaction efficiency, the ratio (b/a) of flow channel length (b) to flow channel diameter (a) is preferably 5 to 200, more preferably 10 to 100, still more preferably 15 to 35.

In terms of reaction efficiency, preferably, an obstacle to the flow of the solutions is disposed in the flow channel. This makes it possible to effectively generate turbulent flows, back flows, cross flows, and the like. The mixing in the flow channel is particularly preferably carried out using a static mixer.

The organic solvent solution and the aqueous solvent solution may be mixed at any mixing ratio; however, the mixing ratio is preferably set such that phospholipase D and the compound represented by formula (B) are used in amounts within the above range.

In the production method of the present invention, after the mixing, the resulting mixture is reacted at an enzyme reaction temperature of phospholipase D. The temperature is not limited as long as it is a temperature at which phospholipase D can exert its activity, and it is usually 20 to 70° C., preferably 30 to 60° C., more preferably 40 to 55° C.

The reaction time is not limited. The production method of the present invention enables efficient reaction, so a larger amount of the target product can be obtained even in a short reaction time. From this viewpoint, the reaction time is preferably 240 min or less, more preferably 30 min to 240 minutes, still more preferably 50 min to 200 min, yet still more preferably 70 min to 150 min.

The reaction can be carried out using various reactors. The reaction can be carried out in a storage tank capable of storing the mixture, or can be carried out using a plug flow reactor. When a storage tank is used, preferably, the mixture is stirred in the tank as necessary. When a plug flow reactor is used, usually, the reaction is carried out while moving the mixture in the tube. When the reaction is carried out in a continuous manner, the flow rate of the mixture in the reactor and the like may be adjusted such that the total residence time in the reactor is preferably within the above-mentioned reaction time range.

After completion of the reaction, the solvent is distilled off, and the product can be isolated and purified by a conventional method such as chromatography or recrystallization. The structure of the product can be identified by elemental analysis, MS (FD-MS) analysis, IR analysis, 1H-NMR, 13C-NMR, or the like.

### EXAMPLES

Hereinafter, the present invention is described in detail with reference to Examples; however, the present invention is not limited to these Examples.

### Example 1

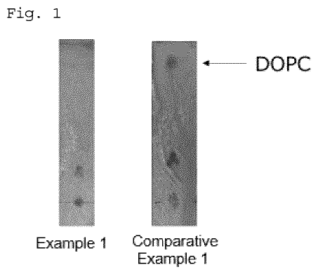

A 280 mL solution of dioleoyl phosphatidylcholine (DOPC) (30 g) in cyclopentyl methyl ether (CPME) was mixed with a 180 mL mixture of 2-(2-aminoethylamino) ethanol (26 g) and phospholipase D (PLDP available from Asahi Kasei Pharma Corporation, 262 U/mg) (60 mg) in an acetate buffer (pH 5.5; 0.5 M) using a static mixer (cross-sectional area of the flow channel: 0.8 mm²; flow rate per mm² of cross-sectional area of the flow channel: 450 cm³/h; ratio (b/a) of flow channel length (b) to flow channel diameter (a): 20). Then, the mixture was sent to a plug flow reactor (50° C.) and a two-stage continuous stirred tank reactor (50° C.) in that order to carry out a continuous reaction. The total residence time in the reactor was 100 minutes. After the raw material solution was consumed, the reaction solution was pushed out with CPME through the plug flow reactor and the two-stage continuous stirred tank reactor in that order, whereby a reaction solution (467 mL in total) was obtained. The reaction solution was analyzed using thin-layer chromatography (TLC) (developing solvent: chloroform/methanol/water=60/30/5; TLC plate used: NH2 silica gel 60 F₂₅₄ plate-Wako (layer thickness 0.25 mm)), whereby the disappearance of DOPC was confirmed (FIG. 1).

The reaction solution was washed with water, extracted, converted to a hydrochloride salt, and re-precipitated to obtain 11 g of a solid (yield 40%). NMR analysis confirmed that the target product (a compound represented by the following formula (DOP-DEDA)) was obtained.

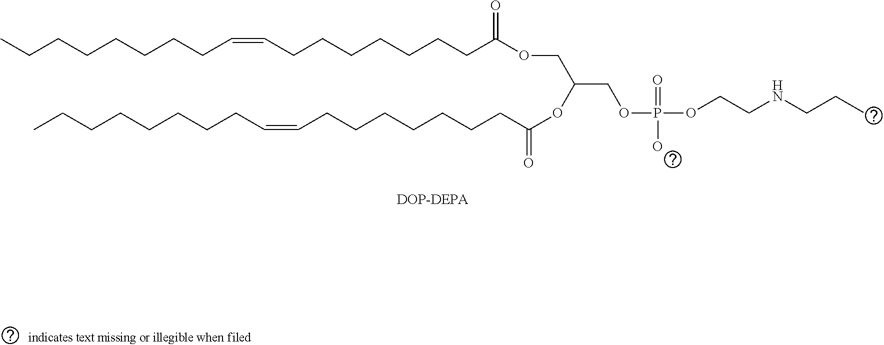 [SMILES: CCCCCCCC/C=C\CCCCCCCC(=O)OCC(COP(=O)(O)OCCNCCC)OC(=O)CCCCCCC/C=C\CCCCCCCC]

### Comparative Example 1

A 6.7 mL DOPC solution in CPME with a concentration adjusted to the same as that in Example 1 and a 4.5 mL solution of 2-(2-aminoethylamino) ethanol and PLDP in an acetate buffer were added to a 20 mL screw tube, and the mixture was stirred at 700 rpm and an internal temperature of 50° C. for 100 minutes using a tapered stirrer (made of PTFE, length 15 mm, diameter 5 mm). After 100 minutes, the stirring was stopped, and the organic layer was subjected to TLC analysis as in Example 1, whereby a DOPC spot was confirmed (FIG. 1).

### Example 2

11 g of DOP-DEDA solid was obtained as in Example 1, except that the static mixer was changed to another mixer capable of mixing two solutions in a flow channel (cross-sectional area of the flow channel: 0.04 mm²; flow rate per mm² of cross-sectional area of the flow channel: appropriately adjustable within the range of 15000 cm³/h to 750000 cm³/h; ratio (b/a) of flow channel length (b) to flow channel diameter (a): 150).

### Example 3

A 225 mL solution of DOPC (30 g) in 4-methyltetrahydropyran (MTHP) was mixed with a 225 mL mixture of 2-[[2-(dimethylamino)ethyl]methylamino]ethanol (47 g) and phospholipase D (PLDP available from Asahi Kasei Pharma Corporation, 260 U/mg) (50 mg) in an acetate buffer (pH 6.0; 0.5 M) using a static mixer (cross-sectional area of the flow channel: 0.8 mm²; flow rate per mm² of cross-sectional area of the flow channel: 450 cm³/h; ratio (b/a) of flow channel length (b) to flow channel diameter (a): 20). Then, the mixture was sent to a plug flow reactor (50° C.) and a two-stage continuous stirred tank reactor (50° C.) in that order to carry out a continuous reaction. The total residence time in the reactor was 100 minutes. After the raw material solution was consumed, the reaction solution was pushed out with MTHP through the plug flow reactor and the two-stage continuous stirred tank reactor in that order, whereby a reaction solution (450 mL in total) was obtained.

The reaction solution was washed with water, extracted, converted to a hydrochloride salt, and re-precipitated to obtain 16 g of a solid (yield 46%). NMR analysis confirmed that the target product (a compound represented by the following formula (compound a)) was obtained.

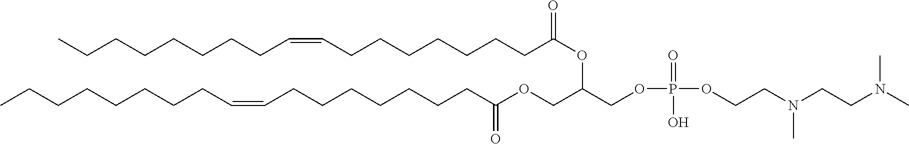 [SMILES: CCCCCCCC/C=C\CCCCCCCC(=O)OCC(COP(=O)(O)OCCN(C)CCN(C)C)OC(=O)CCCCCCC/C=C\CCCCCCCC]

### Example 4

10 g of the compound a was obtained as in Example 3, except that propyl acetate was used instead of MTHP and the static mixer was changed to another mixer capable of mixing two solutions in a flow channel (cross-sectional area of the flow channel: 0.04 mm²; flow rate per mm² of cross-sectional area of the flow channel: appropriately adjustable within the range of 15000 cm³/h to 750000 cm³/h; ratio (b/a) of flow channel length (b) to flow channel diameter (a): 150).

1. A method for producing a phospholipid represented by formula (1) according to reaction equation I, 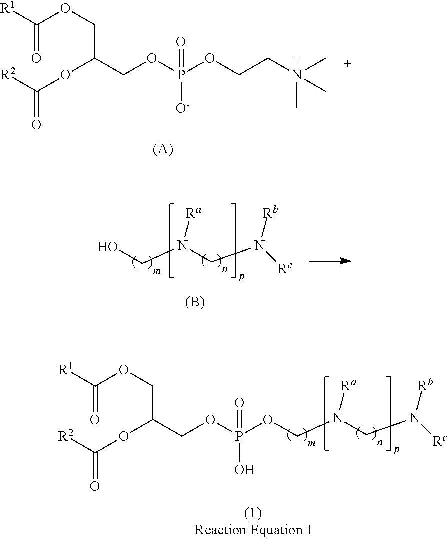
wherein R¹ and R² are the same or different and each represents an acyclic hydrocarbon group; R^{a}, R^{b}, and R^{c} are the same or different and each represents a hydrogen atom or a C₁-C₅ hydrocarbon group; R^{a} and R^{b} may be linked together to form a ring containing adjacent nitrogen atoms; m represents 1 or 2; n represents 1 or 2; and p represents an integer of 1 to 4,
the method comprising:
mixing an organic solvent solution containing a compound represented by formula (A) with an aqueous solvent solution containing phospholipase D and a compound represented by formula (B) in a flow channel; and
reacting the mixture at an enzyme reaction temperature of phospholipase D.

2. The method according to claim 1, wherein a cross-sectional area of the flow channel is 0.01 mm² to 10 mm².

3. The method according to claim 1, wherein a flow rate per mm² of cross-sectional area of the flow channel is 50 cm³/h to 1000000 cm³/h.

4. The method according to claim 1, wherein the mixing in the flow channel is carried out using a static mixer.

5. The method according to claim 1, wherein the organic solvent solution contains an ether solvent and/or an ester solvent.

6. The method according to claim 1, wherein the aqueous solvent solution contains a buffer.

7. The method according to claim 1, wherein the reaction is carried out for 240 minutes or less.

8. The method according to claim 1, wherein at least a part of the reaction is carried out in a plug flow reactor.

9. The method according to any one of claims 1 to 8, wherein the acyclic hydrocarbon group is an unsaturated acyclic hydrocarbon group.

10. The method according to any one of claims 1 to 8, wherein the acyclic hydrocarbon group has 12 to 24 carbon atoms.

11. The method according to any one of claims 1 to 8, wherein m and n are both 2.

12. The method according to any one of claims 1 to 8, wherein p is 1 or 2.

13. The method according to any one of claims 1 to 8, wherein R^{a}, R^{b}, and R^{c} are hydrogen atoms.

14. The method according to any one of claims 1 to 8, wherein R^{a}, R^{b}, and R^{c} are the same or different and each represents a C₁-C₅ hydrocarbon group, R^{a} and R^{b} may be linked together to form a ring containing adjacent nitrogen atoms.
